(19) United States
(12) Patent Application Publication
(10) Pub. No.: US 2026/0130592 A1
(43) Pub. Date: May 14, 2026

(54) Method and Assembly for Analysis of Tumor Tissue

(71) Applicant: SpectroCor Oy, Espoo (FI)

(72) Inventors: Juha Rantala, Espoo (FI); Kalle Kotilahti, Espoo (FI)

(21) Appl. No.: 19/384,035

(22) Filed: Nov. 10, 2025

Related U.S. Application Data
(60) Provisional application No. 63/717,909, filed on Nov. 8, 2024.

(51) Int. Cl. A61B 5/00

(52) U.S. Cl. CPC A61B 5/0075; A61B 5/4836; A61B 5/4848; A61B 5/6847; A61B 5/7275

(57) ABSTRACT

A method and an assembly of assessing potential malignancy of cancerous matter in living tissue, containing cytochrome-c-oxidase in oxidized and reduced states. The method comprises the steps of determining the amount and redox ratio of cytochrome-c-oxidase using real-time Near-Infrared Spectroscopy, and assessing the metabolic nature of the tissue based on the ratio between oxidized and reduced states, respectively of the cytochrome-c-oxidase. The metabolic nature corresponding to the potential malignancy of the cancerous matter. Real-time redox measurement can be used for determining the malignancy of cancer during surgery to determine the need for adjuvant therapies.

### CROSS-REFERENCE TO RELATED APPLICATIONS

This application claims priority to and the benefit of U.S. Provisional Application No. 63/717,909, filed Nov. 8, 2024, the entirety of which is incorporated by reference herein.

### TECHNICAL FIELD

The present invention relates to analysis of tumor tissues. In particular, the present invention concerns a method and an assembly of assessment and analysis of potential cancer malignancy in tumorous tissues.

### BACKGROUND

During cancer surgery, it would be desirable to be able to make real-time assessment and analysis of suspected cancerous tissue. Such assessment and analysis could assist surgeons in determining the aggressiveness of a tumor. Further support could thus be obtained for decisions regarding postoperative treatment protocols, such as the need for adjuvant therapies like chemotherapy or radiation.

Current methods for determining cancer malignancy, such as histopathological analysis or glucose uptake measurements, do not provide real-time feedback on the tumor.

It is an aim of the present invention to provide a method and assembly for real-time assessment and analysis of suspected cancerous tissues.

### SUMMARY OF INVENTION

The present invention provides a system and method for real-time monitoring of cytochrome-c-oxidase (CcO) redox states in living tissues for assessing and analyzing cancer malignancy.

The metabolic flexibility of cancer cells, particularly their ability to switch between oxidative phosphorylation and glycolysis, plays a critical role in tumor malignancy and progression. Cancer cells often favour glycolysis, even in oxygen-rich environments, a phenomenon known as the Warburg effect. This shift reduces the tumor's reliance on oxidative phosphorylation for energy production, which is typically regulated by cytochrome-c-oxidase, the terminal enzyme in the mitochondrial electron transport chain.

Cytochrome-c-oxidase (herein abbreviated “CcO”) enables cells to utilize oxygen in energy production via oxidative phosphorylation, making its activity a critical marker of cellular metabolism. In cancer cells, the ability to sustain CcO activity—measured in real-time—provides essential insights into the tumor's reliance on oxygen for energy generation. Tumors with reduced CcO activity rely predominantly on glycolysis, a hallmark of higher malignancy and more aggressive tumor growth. Reduced CcO concentration in tissue is a reason for reduced CcO activity.

The invention is based on the use of Near-Infrared Spectroscopy for detecting shifts in the redox states of CcO in cancerous tissues.

Thus, in one aspect, the present invention provides a real-time NIRS-based sensor system for measuring cytochrome-c-oxidase redox states to assess cancer malignancy in living tissues.

In another aspect, the present invention provides a method for analyzing cancer malignancy by monitoring cytochrome-c-oxidase redox state changes in real-time, using an NIRS sensor.

More specifically, the present invention is mainly characterized in what is stated in the independent claims.

In some aspects, the present invention enhances the ability to assess malignancy dynamically, offering more precise surgical guidance and improving decisions on post-operative care. The present invention also aids in evaluating tumor responses to chemotherapy by detecting metabolic shifts in the tumor environment.

The real-time data provided offers insights into cellular metabolic behavior, facilitating malignancy assessment and improving diagnostic accuracy. The method and assembly can be used during tumor resection surgeries to identify malignant regions based on metabolic activity. By using spectroscopy, such as Near-Infrared Spectroscopy (in the following also referred to under the abbreviation “NIRS”), to measure the redox state of cytochrome-c-oxidase in real-time, clinicians can obtain immediate insights into the tumor's metabolic behavior during surgery.

In contrast to current metabolic imaging techniques that do not generally offer continuous, in vivo data about CcO redox states, aspects of the present invention allow for ongoing analysis during surgical procedures in living tissue, cancer treatments, or biopsies.

In an aspect of the present invention, there is provided a method of assessing potential malignancy of cancerous matter in living tissue containing cytochrome-c-oxidase in oxidized and reduced states, the method comprising:
determining the molarity and redox level of ratio of cytochrome-c-oxidase using real-time Near-Infrared Spectroscopy, and
determining the metabolic nature of the tissue based on the ratio between oxidized and reduced states, respectively of the cytochrome-c-oxidase, said metabolic nature corresponding to the potential malignancy of the cancerous matter.

In another aspect, there is provided a method of assessing potential malignancy of cancerous matter in living tissue containing cytochrome-c-oxidase in oxidized and reduced states, the method comprising:
determining a molar ratio between the oxidized and reduced states of the cytochrome-c-oxidase using real-time Near-Infrared Spectroscopy, and
assessing the metabolic nature of the tissue based on the ratio between oxidized and reduced states, respectively of the cytochrome-c-oxidase, said metabolic nature corresponding to the potential malignancy of the cancerous matter.

In another aspect of the present invention, there is provided a method of assessing potential malignancy of cancerous matter in living tissue containing cytochrome-c-oxidase in oxidized and reduced states, the method comprising:
inserting a probe comprising at least two separate, protruding lightguides insertable into the tissue of the subject, wherein a first lightguide is arranged to deliver light and a second lightguide is arranged to collect light, and wherein the first lightguide and the second lightguide are spaced apart; and
determining a molar ratio between the oxidized and reduced states of the cytochrome-c-oxidase in the tissue using real-time Near-Infrared Spectroscopy.

In another aspect of the present invention, there is provided a method of assessing potential malignancy of cancerous matter in living tissue containing cytochrome-c-oxidase in oxidized and reduced states, the method comprising:
inserting a probe comprising at least two separate, protruding lightguides insertable into the tissue of the subject, wherein a first lightguide is arranged to deliver light and a second lightguide is arranged to collect light, and wherein the first lightguide and the second lightguide are spaced apart; and
determining a molarity and redox ratio of cytochrome-c-oxidase in the tissue using real-time Near-Infrared Spectroscopy.

### BRIEF DESCRIPTION OF DRAWINGS

FIG. 1 is a schematic depiction of an assembly according to first embodiment, comprising a light source, light detector, and a computing unit;

FIG. 2 is a schematic depiction of an assembly according to a second embodiment, comprising a light source, light detector, lightguides, monitor and a computing unit;

FIG. 3 is a schematic depiction of an assembly according to a third embodiment, comprising a light source, light detector, lightguides, monitor and a computing unit, in which lightguides delivering photons from the light source are not directly connected to the light source and lightguides delivering photons collected from the measured tissue are not directly connected to the light detector;

FIG. 4 is a schematic depiction of an assembly according to a fourth embodiment, comprising a light source, light detector, lightguides, monitor and a computing unit, wherein the lightguides are parallel to each other and separated from each other by a distance and a first lightguide is adapted to direct light against a tumor and a second lightguide is adapted to collect light from the tumor;

FIG. 5 is a schematic depiction of an assembly according to a fifth embodiment, comprising a light source, light detector, lightguides, monitor and a computing unit, wherein the lightguides are parallel to each other and separated from each other by a distance, inserted into a tumor, and a first lightguide being adapted to emit light inside the tumor and a second lightguide being adapted to collect light from inside the tumor;

FIG. 6 is a schematic depiction of an assembly according to a fifth embodiment, comprising a light source, light detector, lightguides, monitor and a computing unit, wherein the lightguides are inserted into a tumor from opposite sides, and a first lightguide is adapted to direct light in one direction inside the tumor and a second lightguide is adapted to collect light from the opposite direction inside the tumor, respectively;

FIG. 7 is a schematic depiction of an assembly according to a sixth embodiment, comprising a light source, light detector, lightguides, monitor and a computing unit, wherein the lightguides are directed towards a tumor from opposite sides, one lightguide being adapted to direct light in one direction against the tumor and a second lightguide being adapted to collect light from the opposite direction outside of the tumor;

FIG. 8 is a schematic presentation illustrating how the ability of a tumor to use oxidative phosphorylation/oxygen can be measured and analyzed.

### DESCRIPTION OF EMBODIMENTS

In the present context, radiation in the range of about 600 to about 900 nm is considered to be radiation in the “near-infrared range”. Spectroscopy using light in the range of 600 to 900 nm can be referred to Near-Infrared Spectrometry (abbreviated “NIRS”).

The molar ratio between the oxidized and reduced states of cytochrome-c-oxidase is referred to as “the redox state” of cytochrome-c-oxidase.

The redox state can be expressed in terms of the ratio of molar concentrations (calculated in mol/l) of cytochrome-c-oxidase molecules present in oxidized and reduced states, respectively, as measured by spectrometry using NIR.

Thus, the redox ratio (state) is, in the present context, expressed as:

$$[ oxidized cytochrome - c - oxidase ( mol / l ) ] / [ reduced cytochrome - c - oxidase ( mol / l ) + oxidized cytochrome - c - oxidase ( mmol / l ) ] .$$

The present invention uses spectroscopy measurement to detect the redox state of CcO in real time in a tissue of interest, such as a suspected cancerous tissue. The device is adapted to capture differences in light absorption linked to the oxidized or reduced states of CcO, reflecting the potential cancer cells' reliance on oxidative phosphorylation or glycolysis. As discussed above, any shifts between the oxidized and reduced states of CcO can indicate the metabolic nature of the tissue. For example, tissue with predominantly reduced CcO could be undergoing aerobic glycolysis (Warburg effect), typically associated with higher malignancy.

Generally, a redox ratio of CcO of less than 50:50, in particular less than 40:60, such as less than 30:70 or less than 20:80 will indicate an increased malignancy of the cancerous tissue. Thus, a redox ratio of CcO of 10:90 or less is considered as a clear indicator of a malignant tumor.

In a first embodiment, a real-time sensor system is used in a method for measuring cytochrome-c-oxidase redox states to assess cancer malignancy in living tissues.

In an embodiment, the system comprises a sensor and the sensor is configured to detect wavelengths of light that correspond to the absorption coefficients of oxidized and reduced forms of cytochrome-c-oxidase.

In some embodiments, the sensor detects changes in wavelengths in the range of about 600 to about 900 nm, in particular in wavelengths of 605 nm, 630 nm, and/or 825 nm to measure the amount of cytochrome-c-oxidase, and differentiate its oxidized and reduced forms. In one embodiment, the light used comprises only one wavelength (monochromatic light), such as 605 nm, 630 nm or 825 nm. In one embodiment, the light comprises several wavelengths, for example, polychromatic light, in one particular embodiment, having at least one peak at 605 nm, 630 nm or 825 nm. In one embodiment, the light source is selected from one or more LEDs. In another embodiment, the light source is selected from a laser, such as a low-level (or low energy) lasers.

In the present technology, the term “sensor” or “probe” designates any suitable structure or method for collecting and/or analysing light obtained from a tissue of interest. Typically, the sensor or probe comprises one or more lightguides (a lightguide for ease of reference) and one or more light collectors (“a light collector” for ease of reference).

In the present disclosure, the spectrometer is generally capable of measuring at least radiation having one wavelength. In case of polychromic light, the spectrometer is capable of separating and measuring spectral components of the light.

In some embodiments, the spectrometer has a probe formed by a lightguide. Such a lightguide can comprise one optical fibre or a bundle of optical fibres. Generally, the lightguide is connected to a light source and exhibits a light emitting first distal end for directing light towards the tissue of interest.

The spectrometer probe is placed into, on, or near the tissue to be analyzed to allow for light being directed towards the tissue of interest.

In one embodiment, the spectrometer comprises a probe with at least two separate protruding lightguides, at least one first lightguide and at least one second lightguide. The lightguides are insertable in a tissue of interest such that a first lightguide is arranged to deliver light and a second lightguide is arranged to collect light.

In one embodiment, the spectrometer comprises one or more probes (hereinafter “a probe”) with at least two separate protruding lightguides, at least one first lightguide and at least one second lightguide. The lightguides are insertable in a tissue of interest such that a first lightguide is arranged to deliver light and a second lightguide is arranged to collect light.

The first lightguide and the second lightguide are spaced apart, such that once they are inserted into the tissue of interest at an insertion point, at least a portion of intact tissue separates the first lightguide from the second lightguide to ensure that light from the first lightguide passes through the tissue on its path to the second lightguide.

For assisting the insertion of the probe through the skin of the tissue of interest, in one embodiment, the probe comprises an insertion aid device. Thus, one or several hollow tube(s) or one or several protecting and/or supporting sleeve(s) can be used for guiding the probe to a predetermined location, and the lightguides are then passed through the tube(s) or sleeve(s) for the measurement, for example, by sliding movement inside the tube(s) or sleeve(s). Typically, the tube(s) or sleeve(s) have a stiffness greater than that of the lightguides.

It is also possible to make the lightguides stiff as such or to provide the lightguides with a covering increasing stiffness, such that they can be inserted into the area of interest separately or together without the use of separate insertion aid devices.

In one embodiment, a majority of the light emitted from the first lightguide and received by the second lightguide will have travelled through intact tissue, and typically less than 10%, in particular less than 5%, preferably less than 1% of the light received by the second lightguide will have travelled through any superficial tissue.

In one embodiment, the mutual distance between the first lightguide and the second lightguide is between 0.1 mm and 5 mm, preferably between 1 mm and 2 mm.

In some embodiments, the insertion depth of at least one of the lightguides is advantageously about 1 mm-10 mm, preferably from 2 to 8 mm, for example, from 4 mm to 6 mm. In one embodiment, the lightguides are inserted in parallel and equally deep into the tissue.

In some embodiments, the lightguides are connected to a control unit for operating the spectrometer. Thus, the control unit may be arranged to generate light in at least one first lightguide for delivering the light to the tissue under monitoring, e.g., to the suspected cancerous tissue. To that end, in some embodiments, the control unit is provided with a light source (a lamp). At least one second lightguide is arranged to collect light from the tissue under monitoring and deliver it back to the control unit for performing an analysis—at least on the basis of the delivered and collected light.

The light fed through the first lightguide into the tissue of interest is selected from light in the range discussed above. In particular, the light has a wavelength in the range of about 600 to about 900 nm, such as 605 nm, 630 nm, and/or 825 nm.

To prevent ambient light from interfering with the measurement, in some embodiments, the probe is provided with a light cover, such as a sheet of non-transparent (opaque) material, to shield off ambient light from the tissue when the lightguides of the probe are inserted into the tissue of interest. In some embodiments, the light cover comprises a material which is tissue confirming.

In one embodiment, the light cover comprises a sheet of flexible polymeric material, such as silicone. In one embodiment, the light cover is attached to a protecting or supporting tube or sleeve. In one embodiment, the light cover is combined with a protecting or supporting tube or sleeve into an integral part.

In one embodiment, an assembly for real-time Near-Infrared Spectroscopy of assessing potential malignancy of cancerous matter in living tissue is disclosed, which comprises:
a probe with at least two separate, protruding lightguides insertable into the cancerous matter of the living tissue at an insertion point, wherein a first lightguide is arranged to deliver light and a second lightguide is arranged to collect light, and wherein the first lightguide and the second lightguide are spaced apart;
a light cover formed by a non-transparent sheet for shielding off ambient light about the insertion point,
a light source, adapted to provide light having a wavelength in the range of 600 to 900 nm, connected to the first lightguide; and/or
a spectrometer connected to the second lightguide configured to provide online spectroscopy analysis of the collected light.

In one embodiment, intraoperative measurement of Cytochrome-C-Oxidase in real-time, employing a spectrometer, is carried out as follows:
1. A probe (which may also referred to herein as “a light collector”) is placed into, on or near the tissue to be analyzed.
2. Near-infrared light is passed through the tissue, and the probe detects changes in the absorption spectra related to the amount of CcO and its redox state.
3. Data is continuously monitored, providing real-time insights into the tissue's metabolic state.

To carry out the measurement, an assembly is used, which, for example, can be of an embodiment shown in the attached drawings.

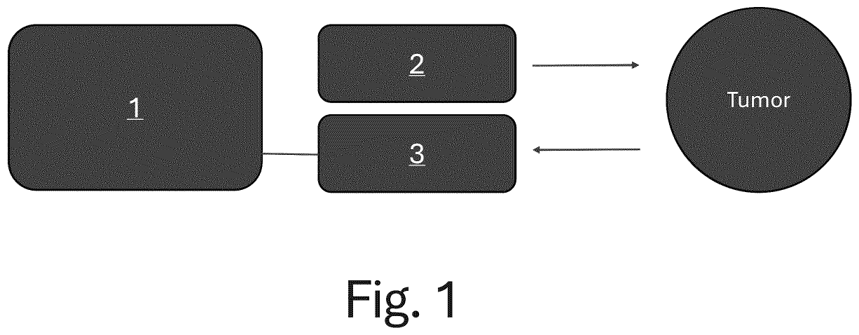

FIG. 1 shows the basic set up of an assembly according to a first embodiment, the assembly comprising a light source 2 generating NIR light, a light detector 3 for detecting NIR light and for conducting it to a computing unit 1. The light detector 3 includes a spectrometer (not separately shown).

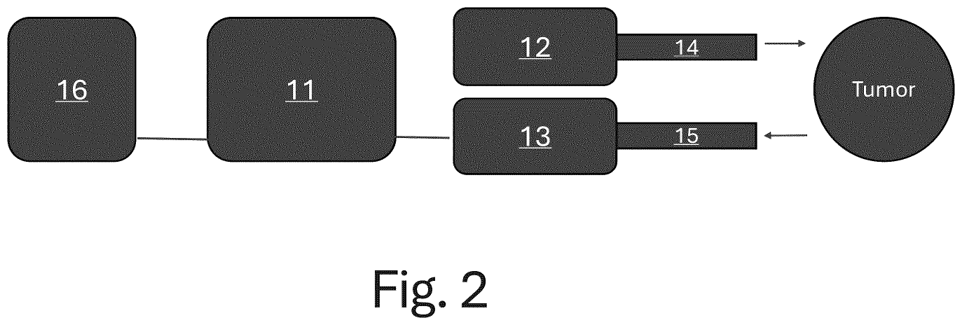

FIG. 2 shows an assembly according to a second embodiment, comprising a light source 12, a light detector 13, and two lightguides of which one (no. 14) which is also referred to as “a first lightguide” is connected to the light source 12, whereas another 15, which is also referred to as “a second lightguide”, is connected to a light detector 13. The assembly further comprises a monitor 16 and a computing unit 11.

As will be evident, the light source 12 and the first lightguide 14 are configured for emitting and directing near-infrared light (photons) to and through the tissue of interest.

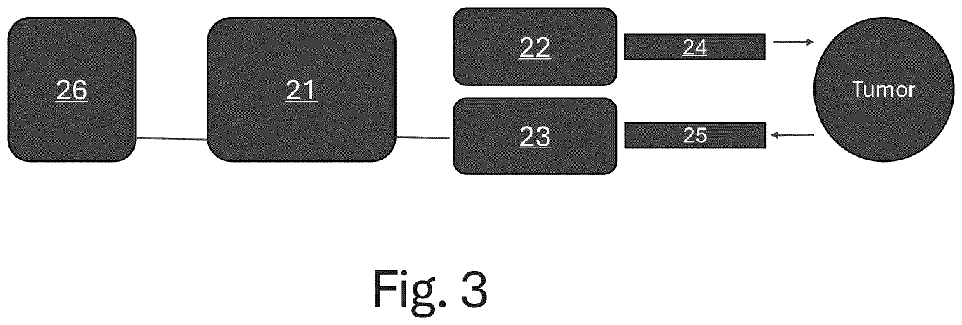

FIG. 3 is a schematic depiction of an assembly according to a third embodiment, the assembly comprising a light source 22, a light detector 23, lightguides 24, 25, a monitor 26 and a computing unit 21. The lightguide 24 delivering the photons emitted from the light source 22 are, in this embodiment, not directly connected to the light source and the lightguide 25 delivering the photons collected from the measured tissue to the light detector are not directly connected to the light detector 23. The lack of a direct connection to the light source enables to use changing or tunable light sources and the lack of direct connection to the light detector enables use of a tunable light detector.

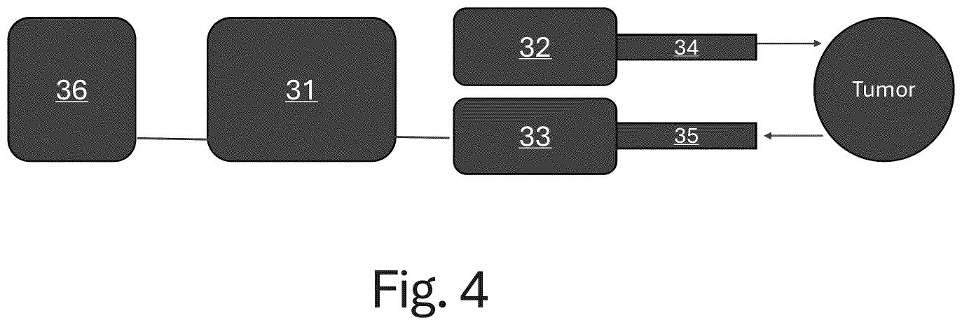

FIG. 4 shows an assembly according to a fourth embodiment, the assembly comprising a light source 32, a light detector 33, lightguides 34, 35, a monitor 36 and a computing unit 31, wherein the lightguides 34, 35 are adapted in a parallel position to each other and separated from each other by a distance, as discussed above. The first lightguide 34 is adapted to direct emitted light towards the surface of a tumor and the second lightguide 35 is adapted to collect light from the tumor.

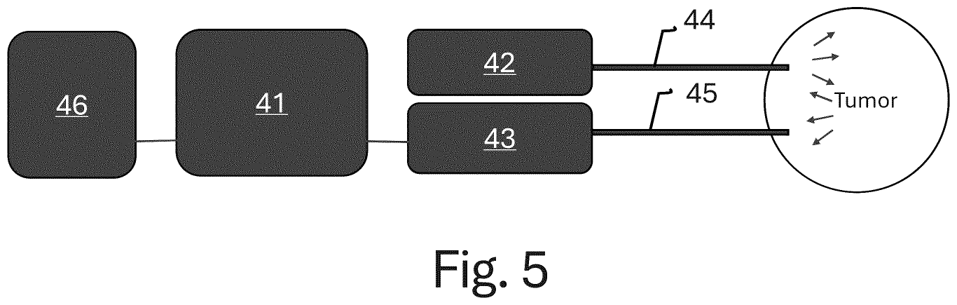

FIG. 5 shows an assembly according to a fifth embodiment, the assembly comprising a light source 42, a light detector 43, lightguides 44, 45, a monitor 46 and a computing unit 41, wherein the lightguides 44, 45 are in parallel to each other, separated from each other by a distance, and inserted into a tumor. Typically, the ends of the lightguides are inserted into the tumor tissue to a depth of at least 1 mm. The first lightguide 44 is adapted to emit light (i.e. photons) inside the tumor and the second lightguide 45 is adapted to collect light from inside the tumor.

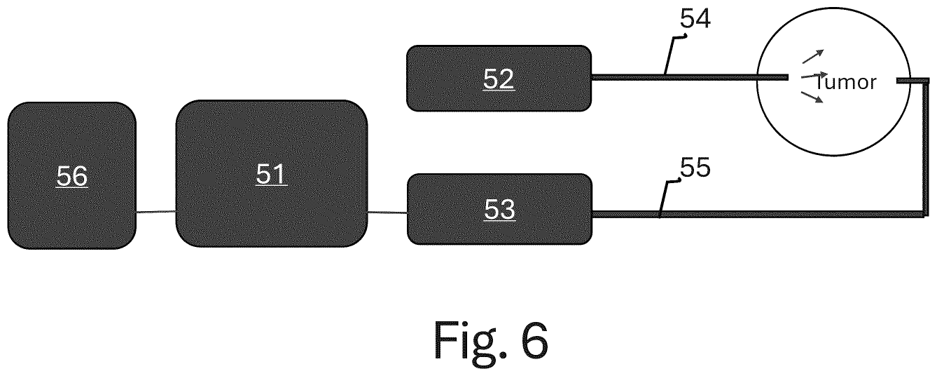

FIG. 6 shows an assembly according to a sixth embodiment, the assembly comprising a light source 52, a light detector 53, lightguides 54, 55, a monitor 56 and a computing unit 51. In this embodiment, the lightguides 54, 55 are inserted into a tumor from opposite sides. The first lightguide 54 is adapted to direct light in one direction inside the tumor and the second lightguide 55 is adapted to collect light from the 2 opposite direction inside the tumor.

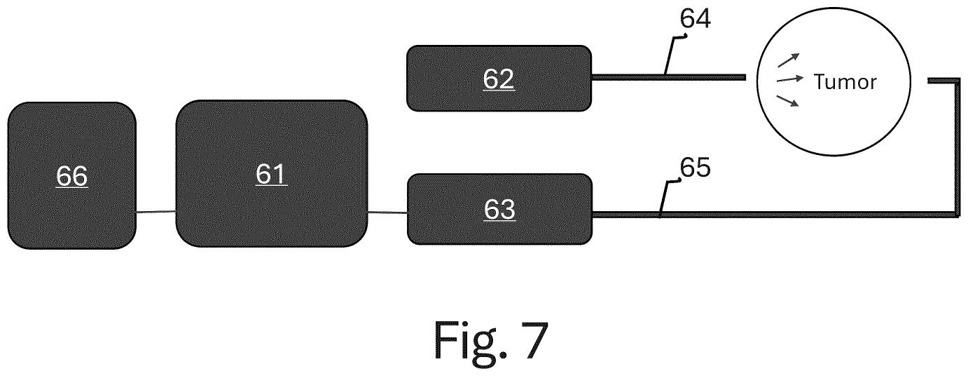

FIG. 7 shows an assembly according to a seventh embodiment, the assembly comprising a light source 62, light detector 63, lightguides 64, 65, a monitor 66 and a computing unit 61. In this embodiment, lightguides 64, 65 are directed towards a tumor from opposite sides, the first lightguide 64 being adapted to direct light in one direction against the tumor, from the outside of the tumor, and the second lightguide 65 being adapted to collect light from the opposite direction outside of the tumor.

In each of the above embodiments, data is continuously monitored, providing real-time insights into the tissue's metabolic state. More specifically, the monitor 3, 16, 26, 36, 46, 56 and 66 is adapted to display real-time data, computed by the computational unit 1, 11, 21, 31, 41, 51 and 61, on redox ratio of CcO, based on the spectral data provided by the light collector 3, 13, 23, 33, 43, 53 and 63.

In one embodiment, the real-time data obtained by measuring the redox state of cytochrome-c-oxidase in the tissue of interest using spectroscopy is used to assist in surgical decision-making, such as identifying malignant regions during tumor resection.

In one embodiment, the data obtained as disclosed herein is processed to track tumor response to chemotherapy, or other treatments by observing metabolic changes in tumor tissues.

In an embodiment, real-time feedback is provided by continuously monitoring the redox state and displaying the data via a connected monitor or an AI-driven platform that correlates redox changes to tumor aggressiveness.

In an embodiment, the tumor's oxygen-utilization capacity is analyzed by changing the oxygenation levels of the patient. By, for example, administering 100% oxygen during surgery, it can be determined whether the tumor's cytochrome-c-oxidase redox state is improved in response to increased oxygen availability.

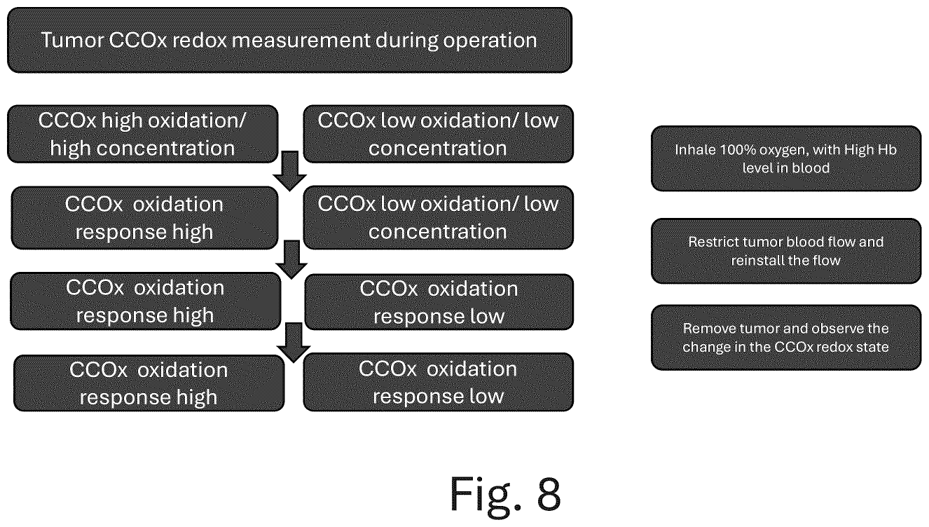

In one embodiment for measuring and analysing the ability of a tumor to use oxidative phosphorylation/oxygen, as shown in FIG. 8, a first determination of the ratio between the oxidized and reduced states of the cytochrome-c-oxidase is carried out using real-time Near-Infrared Spectroscopy, typically with the modified Beer-Lambert law during a first period of time, then the oxygenation levels of the living tissue is increased, e.g. by using inhaled oxygen gas concentration of over 50%, preferably 70 to 100% and/or increasing hemoglobin concentration of incoming blood at least over 10 g/L, preferably over 20 g/L, and a second determination of the ratio between the oxidized and reduced states of the cytochrome-c-oxidase is carried out when a new plateau level of cytochrome-c-oxidase redox status has been reached, using real-time Near-Infrared Spectroscopy during a second period of time. Reaching a plateau takes from 1 to 10 minutes, but a plateau reached during 60 min can be used.

The difference between the ratio between the oxidized and reduced states of the cytochrome-c-oxidase obtained during the first and the second periods of time is indicative of the response of the living tissue to the increased oxygen availability and hence to the potential malignancy of the cancerous matter. The change in the measured redox state can be used to estimate malignancy of the tissue.

As mentioned above, generally, a redox ratio of CcO of less than 50:50, in particular less than 40:60, such as less than 30:70 or less than 20:80 will indicate an increased malignancy of the cancerous tissue. Thus, a redox ratio of CcO of 10:90 or less is considered as a clear indicator of a malignant tumor.

A tumor that does not improve its redox state under high oxygen conditions of more than 15 to 20 Torr may be more glycolytic and, thus, more malignant. Conversely, if the tumor shows an increased redox state with increased oxygen, this may indicate a greater ability to use oxidative phosphorylation, suggesting a less aggressive metabolic profile.

Further, with respect to the embodiment of FIG. 8, in one embodiment, the assembly (in the disclosure also “device”) is configured to establish a baseline measurement of CcO redox state in situ within the tumor. This baseline measurement represents the tumor's existing oxidative phosphorylation activity under normal physiological conditions. The equations may take into consideration the CcO concentration in tissues.

After establishing the baseline redox state of CcO, the assembly is adapted to carry out steps to increase oxidative phosphorylation. This can be reached by increasing oxygen availability in the measured tissue. For example, oxygen availability may be enhanced through supplemental oxygen or controlled hyperoxia by administering 100% oxygen, or hyperbaric oxygen to the patient. This increased oxygen delivery stimulates mitochondrial oxidative phosphorylation, which is reflected in changes to the CcO redox state.

Alternatively, blood flow to the tumor can be augmented through increasing the blood flow by vasodilation techniques or increased tissue perfusion. Such maneuvers provide additional oxygen and substrates, potentially altering the tumor's redox state if the cells can use oxidative phosphorylation. The assembly continuously monitors the redox state of CCOx, and records change in comparison to the baseline, indicating the tumor's oxidative phosphorylation response.

In one embodiment, to augment blood flow to the tumor site, active substances are used. Examples include vasodilatatory drugs, such as nitric oxide and phosphodiesterases. Further, drugs that increase heart function and cause vasodilatation, so-called inodilatators, can also be used. These include drugs from the classes of phosphodiesterase inhibitors, sympathomimetic drugs, and dopaminergic drugs. Phosphodiesterase inhibitors for this purpose include, for example, bipyridine, imidazolone, and benzimidazole derivatives. Dopaminergic drugs include, for example, dopamine, ibopamine, and dobutamine.

In one embodiment, steps to reduce oxidative phosphorylation are carried out. For this purpose, oxidative phosphorylation inhibitors (such as metformin, nitric oxide, and arsenic trioxide) can be used. The ability/inability of the tumor to response to the inhibitory substances reflect the functionality of the CcO pool in the tissue indicating malignancy.

In yet another embodiment, an assembly or process as disclosed herein applies steps to promote anaerobic stimulation, such as anaerobic ATP production. After establishing baseline oxidative phosphorylation levels, anaerobic ATP pathways can be stimulated. For instance, maneuvers, such as hypoxia induction or ischemic conditioning, may be implemented by temporarily reducing oxygen availability. This drives the tumor cells to rely on glycolysis, thus increasing anaerobic ATP production.

Further anaerobic stimulation may include the administration of glucose or insulin, or both, up to the level of insulin clamp, which enhances glucose availability and cellular uptake, fueling glycolysis. Additionally, steps, such as bicarbonate loading or beta-alanine supplementation, can be applied to buffer intracellular acidosis, allowing glycolysis to continue longer under anaerobic conditions. Also, the administration of L-glutamine or its precursor, alpha-ketoglutarate, is another way to alter anaerobic energy production as a response according to the tissue malignancy.

In one additional embodiment, the assembly's computing unit analyzes data to compare the oxidative phosphorylation response (measured through CcO redox changes) with the anaerobic ATP response (measured under hypoxic and glycolytic-promoting conditions). This comparative analysis enables an assessment of the metabolic behavior of the tumor. Tumors with a higher response to anaerobic conditions may rely predominantly on glycolysis, which can be indicative of higher malignancy. Conversely, tumors that demonstrate significant increases in oxidative phosphorylation may exhibit lower malignancy characteristics.

In a still further embodiment, the computing unit is configured to output the results of the CcO redox state analysis in a quantifiable metric or graphical format, displaying the balance between oxidative and anaerobic metabolic activity. This metric aids clinicians in determining tumor malignancy and provides guidance on potential post-operative treatments.

In a method according to the embodiment, an additional dynamic testing approach is provided for assessing the tumor's metabolic flexibility during the operation, allowing for real-time adjustment of surgical or post-operative strategies. In some embodiments, the present technology makes it possible to assess cancer malignancy, which is crucial for successful cancer therapy.

The ability to assess the tumor's oxygen utilization in vivo during surgery has far-reaching implications for personalized cancer treatment. Tumors with diminished oxidative phosphorylation capacity, as demonstrated by a poor response to increased oxygenation, may warrant more aggressive adjuvant therapy following resection. More malignant tumors exhibit reduced activity in oxidative phosphorylation, with a corresponding increase in glycolysis. This metabolic reprogramming is often driven by dysfunction in mitochondrial enzymes, such as cytochrome-c-oxidase. By tracking real-time changes in the redox state of CcO, aspects of the present disclosure can help determine the aggressiveness of the cancer, improving diagnostic precision and treatment efficacy.

On the other hand, tumors that retain a higher degree of mitochondrial function may require less intensive treatment, aligning with a personalized medicine approach where therapy is tailored to the tumor's metabolic characteristics.

During tumor resections, the methods and devices disclosed herein can be used to analyze in vivo the ability of the tumor to use oxygen by cytochrome-c-oxidase.

In addition, the methods and devices disclosed herein may guide the surgeon by highlighting areas of low cytochrome-c-activity, which may indicate malignancy.

During tumor resections, the methods and devices disclosed herein may help guide the surgeon by highlighting areas of high metabolic activity, which may indicate malignancy.

Real-time redox measurement can be used to monitor the effects of chemotherapy or radiation therapy, allowing for adjustments in treatment based on metabolic changes.

In some embodiments, the patient is subjected to surgery for a tumor after neoadjuvant chemotherapy, and the tumor cytochrome-c-oxidase redox state is measured during the surgery, which will then give real-time information of the tumor metabolic response to the treatment, especially when interpreted in conjunction with histological data.

In less invasive settings, the methods and devices disclosure herein can be used to measure tissue samples in vivo, providing immediate data on the malignancy without waiting for histological analysis.

In some embodiments, during a glioblastoma resection, the methods and devices disclosed herein may highlight regions where CcO is predominantly reduced, indicating highly malignant tissue that requires removal.

In some embodiments, a breast cancer patient undergoing chemotherapy shows a shift in CcO redox states, indicating a positive metabolic response to the treatment.

In some embodiments, a group of colon cancer patients undergo operation, which shows different activities in the cytochrome-c-oxidase, which can be analyzed in conjunction with histological data and interpreted through large databases, which eventually can lead to individualized postoperative chemotherapy treatment protocols.

In one embodiment, the methods and devices disclosed herein can be used to measure esophageal or gastric tissues determined or suspected to be malignant. The device may be inserted into the esophagus and further to the stomach with the aid of a gastroscope. In these embodiments, lightguides may be inserted through a lumen within the gastroscope, or the lightguides are attached to the side of the gastroscope. The lightguides can be attached to the side of the gastroscope using a plastic or silicone sleeve, which covers both the lightguides and the gastroscope, holding them together.

In another embodiment, lightguides are brought to the site separately, and the gastroscopic device is used to locate the tumor and help to visually set up the measurement in the correct site. Instruments passed through the ports within the gastroscope can be used to locate the sensor at the correct site and position. These instruments can be used to grab the sensor and mechanically aid it in passing the sensor to the desired location.

Such instruments can be forceps, clamps, or loops through which the lightguides are passed. A similar endoscopic system can be used in the rectum and colon using rectoscopic or colonoscopic instruments.

In one embodiment, the lightguides are brought to the suspected tumor using laparoscopic or thoracoscopic methods. In these methods, the lightguides can be passed to the desired location through tubing. Stiff tubing will assist in locating the lightguides to the desired location.

### REFENCE SIGNS LIST

Computational unit 1, 11, 21, 31, 41, 51, 61
Light source 2, 12, 22, 32, 42, 52, 62
Light collector 3, 13, 23, 33, 43, 53, 63
Lightguide (for emitting light) 14, 24, 34, 44, 54, 64
Lightguide (for collecting light) 15, 25, 35, 45, 55, 65
Monitor 16, 26, 36, 46, 56, 66

### Abbreviations

CcO; COOx Cytochrome-C-Oxidase
NIR Near-Infrared
NIRS Near-Infrared Spectroscopy
LED Light Emitting Diode
AI Artificial Intelligence (neural network)

1. A method of assessing potential malignancy of cancerous matter in living tissue containing cytochrome-c-oxidase in oxidized and reduced states, the method comprising:
determining the molarity and redox level of ratio of cytochrome-c-oxidase using real-time Near-Infrared Spectroscopy, and
determining the metabolic nature of the tissue based on the ratio between oxidized and reduced states, respectively of the cytochrome-c-oxidase, said metabolic nature corresponding to the potential malignancy of the cancerous matter.

2. The method according to claim 1, wherein a lower than 50:50 oxidized state of cytochrome-c-oxidase despite availability of oxygen is as an indication of tissue undergoing aerobic glycolysis associated with higher malignancy.

3. The method according to claim 1, comprising detecting changes in wavelengths between 605 nm, 630 nm, and 825 nm to differentiate between oxidized and reduced cytochrome-c-oxidase.

4. The method according to claim 1, wherein the real-time data is used to assist in surgical decision-making, including identifying malignant regions during tumor resection.

5. The method according to claim 1, wherein the real-time data is processed to track tumor responses to chemotherapy or treatments by observing metabolic changes in tumor tissues.

6. The method according to claim 1, wherein real-time redox measurement is used for monitoring the effects of chemotherapy or radiation therapy, allowing for adjustments in treatment based on metabolic changes.

7. The method according to claim 1, wherein the method comprises:
carrying out a first determination of the ratio between the oxidized and reduced states of the cytochrome-c-oxidase using real-time Near-Infrared Spectroscopy during a first period of time,
increasing the oxygenation levels of the living tissue, and
carrying out a second determination of the ratio between the oxidized and reduced states of the cytochrome-c-oxidase using real-time Near-Infrared Spectroscopy during a second period of time,
determining the difference between the molar ratio between the oxidized and reduced states of the cytochrome-c-oxidase obtained during the first and the second periods of time, and
assessing from the difference between the molar ratios, indicating the response of the living tissue to the increased oxygen availability, the potential malignancy of the cancerous matter.

8. The method according to claim 1, wherein the determining step further comprises using a spectrometer probe assembly, comprising:
a probe with at least two separate, protruding lightguides insertable into the living tissue, wherein a first lightguide is arranged to deliver light and a second lightguide is arranged to collect light, and wherein the first lightguide and the second lightguide are spaced apart; and
a light cover formed by a non-transparent sheet for shielding off ambient light.

9. The method according to claim 8, wherein the first lightguide and the second lightguide are spaced apart, such that once they are inserted into the living tissue, at least a portion of intact tissue separates the first lightguide from the second lightguide to ensure that light from the first lightguide passes through the tissue on its path to the second lightguide.

10. The method according to claim 8, wherein the mutual distance between the first lightguide and the second lightguide is between 0.1 mm and 5 mm, and the insertion depth of at least one of the lightguides into the living tissue is from 3 mm-10 mm.

11. The method according to any of the preceding claims, further comprising:
augmenting blood flow to the tumor for altering the tumor's redox state;
continuously monitoring the redox state of cytochrome-c-oxidase; and
recording changes in the redox state of cytochrome-c-oxidase in comparison to a baseline redox state, indicating the tumor's oxidative phosphorylation response.

12. The method according to claim 11, further comprising using substances for augmenting blood flow to the living tissue, such substances being selected from vasodilatatory drugs or drugs that increase heart function and/or cause vasodilatation.

13. An assembly for real-time Near-Infrared Spectroscopy of assessing potential malignancy of cancerous matter in living tissue, comprising:
a probe with at least two separate, protruding lightguides insertable into the cancerous matter of the living tissue at an insertion point, wherein a first lightguide is arranged to deliver light and a second lightguide is arranged to collect light, and wherein the first lightguide and the second lightguide are spaced apart;
a light source, adapted to provide light having a wavelength in the range of 600 to 900 nm, connected to the first lightguide; and
a spectrometer connected to the second lightguide for providing online spectroscopy analysis of the collected light.

14. The assembly according to claim 13, wherein at least a portion of at least one of the lightguides are contained within a protecting and/or supporting sleeve, from which it can be released to allow for insertion into a tissue of interest in particular by a sliding movement.

15. The assembly of claim 13, further comprising a light cover formed by a non-transparent, preferably flexible, sheet for shielding off ambient light about the insertion point.

16. The assembly according to claim 13, wherein the light cover is attached to any protecting or supporting tube or sleeve; in particular the light cover is combined with any protecting or supporting tube or sleeve into an integral part.

17. A method according to claim 1, where concurrent tissue oxidation level is determined by measuring hemoglobin and the cytochrome-c-oxidation redox level is analyzed in comparison.

18. A method of assessing potential malignancy of cancerous matter in living tissue containing cytochrome-c-oxidase in oxidized and reduced states, the method comprising:
inserting a probe comprising at least two separate, protruding lightguides insertable into the tissue of the subject, wherein a first lightguide is arranged to deliver light and a second lightguide is arranged to collect light, and wherein the first lightguide and the second lightguide are spaced apart; and
determining a molarity and redox ratio of cytochrome-c-oxidase in the tissue using real-time Near-Infrared Spectroscopy.
